(19) United States
(12) Patent Application Publication
(10) Pub. No.: US 2021/0267471 A1
(43) Pub. Date: Sep. 2, 2021

(54) SYSTEM AND METHOD FOR EVALUATING A VARIATION OF A HEART RATE OF A SUBJECT

(71) Applicant: KONINKLIJKE PHILIPS N.V., EINDHOVEN (NL)

(72) Inventors: Alberto Giovanni Bonomi, Eindhoven (NL); Alphonsus Tarcisius Jozef Maria Schipper, Stramproy (NL); Jenny Margarito, Eindhoven (NL); Helma Majella de Morree, Waalre (NL)

(21) Appl. No.: 16/093,454

(22) PCT Filed: Apr. 13, 2017
(86) PCT No.: PCT/EP2017/058948

§ 371 Date: Oct. 12, 2018

(30) Foreign Application Priority Data
Apr. 15, 2016 (EP) 16165576.6

(51) Int. Cl. A61B 5/024; A61B 5/00; A61B 5/11; A61B 5/0205

(52) U.S. Cl. CPC A61B 5/02405; A61B 5/02416; A61B 5/7221; A61B 5/1118; A61B 5/1116; A61B 2562/0219; A61B 5/02438; A61B 5/681; A61B 5/0205; A61B 5/0024; A61B 5/1114

(57) ABSTRACT

The present invention relates to a detection of physiological signals of cardiovascular systems. In particular, it relates to a system and method for more reliably evaluating a variation of a heart rate of a subject. The system comprises a photoplethysmography (PPG) signal providing unit (10); a motion signal providing unit (20); a motion determination unit (40) for determining a motion period during which motion identifiable in the motion signal corresponds to one of a plurality of predefined motion classes; a signal reliability determination unit (50) for determining a signal reliability of the PPG signal depending on a motion influence period, wherein the motion influence period comprises the motion period and a transition period following the motion period; and a heart rate variation determination unit (60) for determining the variation of the heart rate of the subject based on the signal reliability and on the PPG signal.

### FIELD OF THE INVENTION

The present invention relates to a detection of physiological signals of cardiovascular systems. In particular, it relates to a system and method for evaluating a variation of a heart rate of a subject. It finds application in diagnostic investigation of arrhythmias, in particular in the detection of atrial fibrillation. However, it is to be understood that the present invention also finds applications in other fields and is not necessarily limited to the above mentioned application.

### BACKGROUND OF THE INVENTION

Cardiovascular diseases are a class of diseases that involve the circulatory systems such as the heart, the blood vessels including arteries, capillaries and veins. One of the most common groups of conditions belonging to cardiovascular diseases is arrhythmia, which is characterized by abnormal heart rates. A patient or a subject suffering from arrhythmia may have a heart beat that is too fast or too slow, wherein the heart beat may be regular or irregular. Arrhythmias can occur in the upper chambers of the heart (atria) or in the lower chambers of the heart (ventricles), one widespread example of arrhythmias is atrial fibrillation (AF).

However, screening for AF is problematic because of the asymptomatic and paroxysmal nature of the condition, especially in the early phase of development. Accordingly, episodic electrocardiography (ECG) measurements are in many cases of limited value. Systems like a smart watch device capable of detecting AF with high diagnostic yields given the long term monitoring capabilities represent unobtrusive, discrete and inexpensive solutions. The wearable monitoring unit allows patients to be monitored throughout the day and night for a long period of time in order to continuously track rhythm disorders onset.

For example, a wrist wearable pulse plethysmographic (PPG) sensor can offer an alternative to electrocardiography (ECG)-based solutions to track cardiac rhythm by processing the time series of interbeat intervals (IBI) and determine AF likelihood. Using PPG sensors for AF episodes identification comes along with the problem that motion artifacts impact the recorded signal and reduce the accuracy of the system.

U.S. Pat. No. 8,974,396 B1 discloses a method for determining a premature atrial contraction event from a PPG signal of a subject and for validating the premature atrial contraction event by measuring motion activity and verifying the validity of the premature atrial contraction event when the motion measured by the accelerometer remains less than a preset threshold.

WO 2015/189304 A1 discloses a heart rate monitor system comprising an inactivity determining unit for determining periods of inactivity of a user based on motion data detected by at least one motion sensor attached to the user and a resting heart rate calculating unit for calculating a resting heart rate of the user based on heart rate data detected by at least one heart rate sensor attached to the user during the periods of inactivity as determined by the inactivity determining unit is provided.

US 2012/0123226 A1 discloses method for operating a monitoring system for monitoring physiological data of a patient. For saving power, motion activity data of the patient is obtained and the measurement of physiological data of the patient is initiated, if the motion activity is non-zero and below a selected threshold.

Yet, comparing the motion with a preset threshold does not satisfactorily account for the reliability of the PPG signal due to the influence of motion and could potentially lead to false positive identification of premature atrial contraction events as AF events.

### SUMMARY OF THE INVENTION

It is thus an object of the present invention to provide a system and a method, which allow for a more reliable evaluation of variations of a heart rate of a subject.

In a first aspect of the present invention a system for evaluating a variation of a heart rate of a subject is provided, comprising: a PPG signal providing unit for providing a PPG signal indicative of a heartbeat of the subject; a motion signal providing unit for providing a motion signal indicative of a motion of the subject; a signal feature determination unit for determining a signal feature of the PPG signal, wherein the optical feature is at least one of an interbeat interval (IBI) or a morphology of the PPG signal; a motion determination unit for determining a motion period during which motion identifiable in the motion signal corresponds to one of a plurality of predefined motion classes; a signal reliability determination unit for determining a signal reliability of the PPG signal depending on whether a period of the PPG signal is within or outside a motion influence period, wherein the motion influence period comprises the motion period and a transition period following the motion period; and a heart rate variation determination unit for determining the variation of the heart rate based on the signal reliability and on the signal feature of the PPG signal.

Since the heart rate variation determination unit determines the variation of the heart rate based on the signal reliability of the PPG signal, periods of the PPG signal, which are unreliable or have a low reliability, for instance those which are within the motion influence period, can be left unconsidered. In other words, time periods of the PPG signal for which the signal reliability determination unit determines a low reliability can be left unconsidered or contribute with a lower coefficient to the heart rate or the variation thereof. Accordingly, variations of the heart rate are assessed based on a more reliable signal and thus eventually evaluated more reliably.

The heart rate variation determination unit can in one embodiment be adapted to first determine the variation of the heart rate of the subject based on the PPG signal and then to evaluate the determined variation of the heart rate based on the signal reliability. For instance, in an embodiment the signal reliability determination unit can also be adapted to determine the signal reliability of the PPG signal after the heart rate variation determination unit has determined the variation of the heart rate of the subject. Eventually, in all embodiments, the heart rate variation determination unit will determine the variation of the heart rate of the subject based on the signal reliability and on the PPG signal.

An interbeat interval is determined by detecting consecutive peaks indicative of the heart beat in the PPG signal. The IBI corresponds to the time between two such consecutive peaks. The heart rate directly corresponds to the reciprocal of this IBI, wherein the heart rate variation determination unit is preferably configured to determine the heart rate based on the IBI and statistical methods for evaluating a time series of consecutive IBIs. However, also other features of the PPG signal, such as a morphology of the signal, can be used for evaluating a variation of the heart rate.

Since the signal reliability determination unit determines the reliability based on a motion influence period, also periods which are not subject to motion, i.e. time periods during which a motion corresponding to one of the predefined motion classes cannot be detected, but which however are still under the influence of motion, will be taken into account by the signal reliability determination unit for determining the reliability of the corresponding period of the PPG signal.

This solution is based on the finding that motion of the subject which falls within one of the predefined motion classes will influence the reliability of the PPG signal even after the underlying motion has ended. By determining the reliability, based on the motion influence period, the transition period following the motion period is also considered when determining the reliability of the PPG signal. Advantageously, when determining the variation of the heart rate, signal features of the PPG signal will thus only be considered for the evaluation when no motion influence is present in the underlying signal. In other words, a risk for false positive detection of unnatural variation of the heart rate, e.g. atrial fibrillation (AF), due to motion of the subject can be reduced.

The PPG signal providing unit can be a storing unit, in which the PPG signal is stored already, wherein the PPG signal providing unit can be adapted to provide the stored PPG signal. However, the PPG signal providing unit can also be a receiving unit for receiving a PPG signal from a PPG signal measurement unit such as a PPG sensor and for providing the received PPG signal. Moreover, the PPG signal providing unit can be the PPG signal measurement unit itself, wherein the PPG signal providing unit provides the measured PPG signal. The PPG signal measurement unit preferentially comprises one or more light sources for illuminating a part of the subject's body and one or several detectors for detecting the light from the subject's body, wherein the PPG signal measurement unit is preferentially adapted to determine the PPG signal based on the detected light. In a preferred embodiment the PPG signal measurement unit is a pulse oximeter or a heart beat detector.

Likewise, the motion signal providing unit can be a storing unit, in which the motion signal is stored already, wherein the motion signal providing unit can be adapted to provide the stored motion signal. However, the motion signal providing unit can also be a receiving unit for receiving a motion signal from a motion signal measurement unit and for providing the received motion signal. Moreover, the motion signal providing unit can be the motion signal measurement unit itself, wherein the motion signal providing unit provides the measured motion signal.

Preferentially, the PPG signal providing unit and the motion signal providing unit provide a PPG signal and a motion signal, respectively, which originate from the same body part of the subject, such as the wrist. Preferentially, the PPG signal providing unit is the PPG signal measurement unit and the motion signal providing unit is the motion signal measurement unit and the PPG signal measurement unit and the motion signal measurement unit measure the PPG signal and the motion signal, respectively, at the same body part of the subject, such as the wrist.

Preferentially, the PPG signal and the motion signal are synchronous signals and correspond to the same time periods.

In an embodiment of the system, the motion determination unit is adapted to determine a motion class of the plurality of predefined motion classes for the motion period based on the motion signal, wherein the transition period has a length based on the motion class. Advantageously, depending on the motion class, the transition period can thus be shorter or longer. Even further, the duration of the transition period can advantageously be as short as possible to discard or lower the reliability of as little of the PPG signal as possible yet as much as necessary to ensure the validity of the signal.

Preferentially, the motion determination unit is adapted to determine motion signal characteristics of the motion signal and predefined assignments between a motion signal characteristic and a motion class for determining the motion period and the corresponding one of the plurality of motion classes.

In an embodiment of the system, the motion signal is indicative of a motion of a body part of the subject, and the plurality of predefined motion classes comprises i) an activity of the subject, ii) a posture change of the subject and iii) a movement of an adjacent body part. Preferably, the activity of the subject can be any activity other than sedentary, such as cycling, walking, running etc. The determination of the length of the transition period can therefore advantageously be facilitated, since it can be based on a class of motion, even on a predefined class of motion. Preferably, the length of the transition period can be predefined for each of the predefined plurality of motion classes, respectively.

In an embodiment of the system, the motion signal providing unit comprises at least one of a vibration sensor, a rotation sensor and an acceleration sensor for sensing a vibration, a rotation and/or an acceleration of the subject. The motion signal providing unit is preferentially the motion signal measuring unit and comprises one or more accelerometer sensors for measuring accelerations and movements of the subject's body at one or more positions in one or more directions, wherein the motion signal measurement unit is preferentially adapted to determine the motion signal based on the detected accelerations. A classification of a posture change as the motion class can in one embodiment advantageously be detected by determining changes in average orientation of an accelerometer, preferably a 3D accelerometer, with respect to gravity.

In an embodiment of the system, the motion signal providing unit comprises a network of two or more sensors to be provided at different body parts of the subject. Also, in this embodiment, the sensors can be vibration, rotation or acceleration sensors capable of detecting peculiar motion of nearby body parts of the subject with respect to the body part the PPG signal originates from.

In an embodiment of the system, the photoplethysmography signal providing unit is a PPG sensor and the photoplethysmography signal is a PPG signal of the subject. A PPG sensor is adapted to emit a light of one or more wavelengths onto the skin of a subject, such as at the wrist or the like, and to detect reflected light which is indicative of the heart beat of the subject. More precisely, due to subcutaneous blood flow, the light reflected the PPG sensor can be indicative of the heart beat of the subject. The determination of signal features from the PPG signal is highly dependent on motion of the subject. In the example of a PPG sensor to be worn at the wrist, motions of the subject which have an influence on the reliability of the PPG signal include motions for which the arm or the wrist is moving and those for which the arm/wrist is not moving. More precisely, influence due to motion can be distinguished as movement of the body part where the sensor is located, i.e. the arm movement or the wrist movement, and small movement and vibrations due to adjacent limbs motion, i.e. motion of the hand and/or fingers in this example.

In an embodiment of the system, the heart rate variation determination unit is adapted to determine the heart rate of the subject based on the signal reliability and on the signal feature and to determine the variation of the heart rate of the subject based on the heart rate of the subject. The heart rate variation determination unit in this embodiment can be adapted to determine the heart rate of the subject in the first place and to determine the variation of the heart rate of the subject based on the heart rate of the subject in a later stage. The heart rate variation determination unit can also be adapted to determine the heart rate and/or the variation of the heart rate directly based on the signal reliability of the PPG signal or to determine the heart rate and/or the variation of the heart rate without considering the signal reliability of the PPG signal in the first place and to evaluate the heart rate and/or the variation of the heart rate at a later stage based on the signal reliability of the PPG signal.

In an embodiment of the system, the signal reliability determination unit is adapted to determine a binary reliability or a reliability having intermediate values. In case the reliability is determined as a binary result, the PPG signal can be either reliable or unreliable. In other words, the PPG signal and corresponding signal features are either accepted as reliable or discarded as unreliable. In another embodiment, the reliability has intermediate values. Reliability can thus be continuous or have discrete intermediate values. The signal reliability determination unit can thus be adapted for determining any of the intermediate values for the reliability of the PPG signal and can also be adapted for increasing or lowering the reliability for a particular time period. This can be particularly advantageous in case of multiple signals and/or multiple sensors, wherein, for instance, the sensor having the higher or highest reliability for a particular time period is selected.

In an embodiment of the system, the signal reliability determination unit is adapted to determine a higher reliability of the PPG signal outside the motion influence period than within the motion influence period. Advantageously, the signal not subject to motion influence is thus assigned a higher reliability than the signal under the influence of motion.

In an embodiment using IBIs as the signal feature, the reliability of the PPG signal for the current IBI can be lowered as compared to the previous IBI reliability when movement of adjacent body parts it detected. The heart rate variation determination unit can then be adapted for determining the heart rate based on the IBI values and for evaluating the variation based on the IBI reliabilities using statistical classifiers based on, for instance, Markov Model probability or entropy and IBI variability features.

In an embodiment of the system, the system is incorporated in a device with a housing wearable on one or more body parts of the subject. It is noted that the term “wearable” shall also include the general meaning of the term “portable”. The one or more body parts include all body parts of the human body appropriate for wearing or porting the device, such as the arm, the wrist, the waist, the back, the neck, the leg, the foot, etc.

Advantageously, such a device enables continuous detection of the heart rate variation, independent from the location, such as at home, in a vehicle/train/aircraft, at working place, and the activity, such as working, resting, doing sport, having a meal, of the subject. By wearing the device for a long period of time, a long term study of the heart rate progressing can be realized which may provide the surgical person with accurate information about the development of heart diseases including AF and other types of arrhythmias. Besides, AF-episodes might occur on any time of the day; carrying the device continuously thereby makes it possible to detect even unexpected AF-episodes.

In an embodiment of the system, the system is implemented as a wrist based device. Advantageously, the device comprises a wristwatch-like device, the housing being wearable on one wrist or arm of said subject, containing both a PPG signal measurement unit and a motion signal measurement unit. A subject is used to wearing/porting a wristwatch, which has little impact on the daily activities of the subject.

In an embodiment the system comprises: a second PPG signal providing unit for providing a second PPG signal indicative of the heartbeat of the subject; a second motion signal providing unit for providing a second motion signal of the subject; a second motion class determination unit for determining a second motion period during which motion identifiable in the second motion signal corresponds to one of the plurality of predefined motion classes; a second signal reliability determination unit for determining a second signal reliability of the second PPG signal depending on whether a period of the second PPG signal is within or outside a second motion influence period, wherein the second motion influence period comprises the second motion period and a second transition period following the second motion period; wherein the heart rate variation determination unit is adapted to determine the variation of the heart rate of the subject based on the signal reliability, the second signal reliability, the PPG signal and/or the second PPG signal.

Since the second PPG signal is additionally provided, monitoring time can be increased, even if the PPG signal is unreliable for a particular time period due to motion. This system shows particular advantages in situations, in which motion artifacts are restricted to one of the PPG signal and the second PPG signal only.

In an embodiment of the system, the PPG signal and the motion signal are indicative of a first part of the subject, wherein the second PPG signal and the second motion signal are indicative of a second part of the subject, wherein the first part and the second part are two symmetrical body parts of the subject.

For instance, the PPG signal can be indicative of the left wrist and the second PPG signal can be indicative of the right wrist, or vice versa. Preferably, the system comprises two PPG sensors to be attached to the left wrist and the right wrist, respectively. By positioning the two PPG sensors at the same but opposite body location, a single classification scheme can be developed, which alternatively uses data from the two sensors. Preferably, the two PPG sensors are coupled with motion sensors, respectively. In one embodiment, the PPG signal has good quality for half of the signal period and the second PPG signal has good quality during the other half signal period, for instance, during physical activities involving antiphasic arm movements reflected in periodic and antiphasic body acceleration signals. Advantageously, switching between the two sensors allows doubling the time with good quality optical features and thus allows doubling the monitoring time. Furthermore, in one embodiment, evaluating a variation of the heart rate, such as for detecting AF episodes, during intensive physical activities can thus be allowed, providing physicians with information about AF context related episodes. Advantageously, the most appropriate medical treatment can be determined.

In another embodiment the system comprises two PPG sensors and two motion sensors to be provided at the same or nearby body parts of the subject, such as within the same housing of a wrist-based device. Such system allows for a more reliable evaluation of variations of the heart rate due to the duplicate provision of the sensors.

In an embodiment of the system, the heart rate variation determination unit is adapted to determine the higher one of the signal reliability and the second signal reliability and to decide on one of the PPG signal and the second PPG signal based on the higher one of the signal reliability and the second signal reliability.

Advantageously, only the more reliable among the PPG signal and the second PPG signal is considered such that a risk for false positive determination of abnormal variation of the heart rate, such as a false positive determination of AF episodes, can be reduced.

The signal feature determination unit, the motion determination unit, the signal reliability determination unit and the heart rate variation determination unit can in one embodiment be provided in one or more processors that are arranged in the same or different physical devices. More precisely, the signal feature determination unit, the motion determination unit, the signal reliability determination unit and the heart rate variation determination unit can in one embodiment be provided together with the PPG signal providing unit and/or the motion signal providing unit in a single device or in a different embodiment be distributed over multiple devices.

In an embodiment the signal feature determination unit, the motion determination unit, the signal reliability determination unit and the heart rate variation determination unit are adapted for communicating with the photoplethysmography signal providing unit and/or the motion signal providing unit in a wired or wireless manner as well known in the art. In one embodiment, one, more or all of the signal feature determination unit, the motion determination unit, the signal reliability determination unit and the heart rate variation determination unit are provided at a server, which is adapted to communicate with the rest of the evaluation system by suitable communication means, for instance via the Internet.

In a further aspect of the invention a method for evaluating a variation of a heart rate of a subject is provided, comprising

providing a PPG signal indicative of a heartbeat of the subject;

providing a motion signal indicative of a motion of the subject;

determining a signal feature of the PPG signal, wherein the signal feature comprises at least one of an IBI and a morphology of the PPG signal;

determining a motion period during which motion identifiable in the motion signal corresponds to one of a plurality of predefined motion classes;

determining a reliability of the PPG signal depending on whether a period of the PPG signal is within or outside a motion influence period, wherein the motion influence period consists of the motion period and a transition period following the motion period;

determining a heart rate based on the signal feature; and

evaluating a variation of the heart rate of the subject based on the determined reliability of the PPG signal.

In a further aspect of the invention a computer program for evaluating a variation of a heart rate of a subject is provided, the computer program comprising program code means for causing an evaluation system as defined in claim 1 to carry out the evaluation method as defined in claim 13, when the computer program is run on the evaluation system.

It shall be understood that the system for evaluating a variation of a heart rate of a subject of claim 1, the method for evaluating a variation of a heart rate of a subject of claim 13 and the computer program for evaluating a variation of a heart rate of a subject of claim 14 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 schematically and exemplarily shows an embodiment of an evaluation system for evaluating a variation of a heart rate of a subject,

FIG. 2 schematically and exemplarily shows a watch-like device as an embodiment of the evaluation system shown in FIG. 1,

FIG. 3 schematically and exemplarily illustrates a flow chart of an embodiment of a method for evaluating a variation of a heart rate of a subject,

FIG. 4 exemplarily illustrates a first example of a scheme for assessing a reliability of a PPG signal, and

FIG. 5 exemplarily illustrates a second example of a scheme for assessing a reliability of a PPG signal.

### DETAILED DESCRIPTION OF EMBODIMENTS

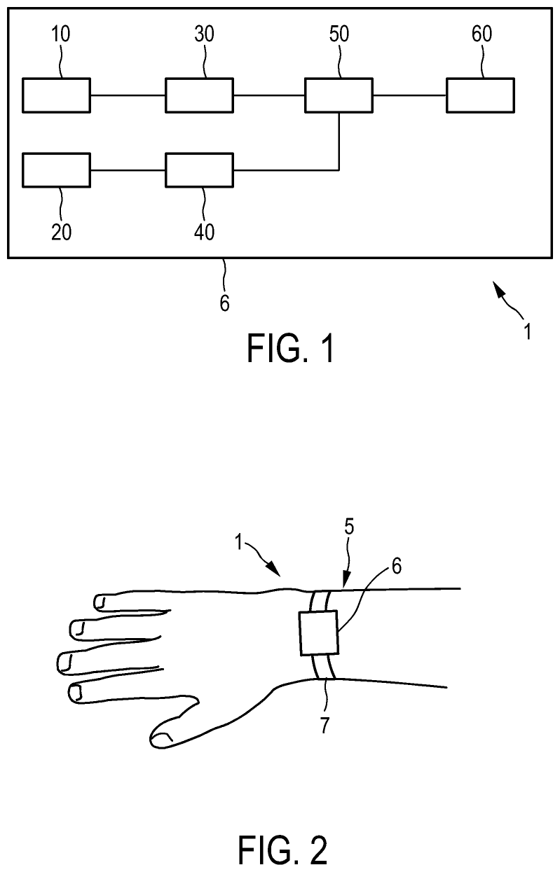

FIG. 1 schematically and exemplarily shows an embodiment of an evaluation system 1 for evaluating a variation of a heart rate of a subject. Evaluation system 1 in this example comprises a casing 6 which houses a PPG signal providing unit 10, a motion signal providing unit 20, a signal feature determination unit 30, a motion determination unit 40, a signal reliability determination unit 50 and a heart rate variation determination unit 60. Although in this example all units are contained within casing 6, in other examples, also one, more or all of the units can be provided at different and/or remote locations, such as implemented on a server.

PPG signal providing unit 10 in this example is a PPG signal measurement unit comprising a PPG sensor for measuring a PPG. This unit can be a known PPG sensor unit, which comprises light-emitting diodes (LEDs) directing light onto the skin of the subject and which detects light reflected from the skin of the subject, wherein PPG sensor unit 10 generates the PPG based on the detected light.

Motion signal providing unit 20 in this example comprises an accelerometer, which detects accelerations of body parts of the subject. In this example, the accelerometer comprises a multi-dimensional accelerometer in order to detect posture changes of the subject by observing changes in average orientation of the accelerometer with respect to gravity. However, in other embodiments, other means and units for providing the motion signal are contemplated.

Signal feature determination unit 30 is arranged for receiving a PPG signal from PPG signal providing unit 10 and for determining a signal feature based on the received PPG signal. In this example, signal feature determination unit 30 determines interbeat intervals (IBIs) from the PPG signal of the subject.

Motion determination unit 40 is arranged for receiving a motion signal from motion signal providing unit 20 and for determining a motion period based on the received motion signal. The motion period is determined by motion determination unit 40 as the period during which a motion detectable in the motion signal falls within a predefined motion class.

In this example, motion determination unit 40 is arranged for evaluating whether one of three motion classes applies: an activity other than sedentary is being detected, which may for example be cycling, walking or running During such activity, IBIs detected by signal feature determination unit 30 cannot reliably be detected from the PPG signal. Motion determination unit 40 is arranged for detecting motion periods by using known pattern recognition techniques on the motion signal, for instance. However, in another example, motion periods can alternatively or additionally be determined by analyzing the magnitude of the motion signal.

Apart from the subject's activities, a posture change can take place and be detected by motion determination unit 40 as motion corresponding to a second motion class. This also prevents proper IBI detection and may be, as indicated above, for example, be detected by observing changes in average orientation of a 3D accelerometer motion signal.

Finally, motion of an adjacent body part can be detected by motion determination unit 40 as motion corresponding to a third motion class. For instance, motion of adjacent parts could be detected by means of sensor networks connecting devices located at the adjacent body parts or by using vibration, rotation or acceleration sensors capable of detecting the peculiar motion pattern of nearby limbs at the location of the PPG sensor.

In one example, adjacent body parts are defined as the body part the motion signal is indicative of In the example of a wrist based device, this could include the wrist and the arm. In another example, adjacent body parts can be defined as limbs or parts of limbs being adjacent to the body part the motion signal is indicative of. In the example of a wrist based device, this could include the hand and the fingers. Motion of the various examples of adjacent body parts can be distinguished based on the motion signal by motion determination unit 40. In even another example, motion determination unit 40 can also be arranged to determine and distinguish motion from multiple or all of the examples of adjacent body parts indicated above.

Signal reliability determination unit 50 is arranged for determining a reliability of the PPG signal depending on whether a period of the PPG signal is within or outside a motion influence period, wherein the motion influence period consists of the motion period determined by motion determination unit 40 and a transition period following the motion period. It receives both the identified signal features from signal feature determination unit 30 and the motion period determined by motion determination unit 40. In case motion determination unit 40 determines motion corresponding to one of the motion classes during a motion period, signal reliability determination unit 50 determines a motion influence period, consisting of the motion period and a transition period which follows the motion period and is indicative of a time period in which no motion can be detected, but which nevertheless is still influencing the reliability of the PPG signal. Signal reliability determination unit 50 can determine the PPG signal during the motion influence period as unreliable or can provide a low reliability of the PPG signal depending on the motion pattern.

All three classes of motion determined by motion determination unit 40 can lead to a lower reliability of signal features determined by signal feature determination unit 30 and/or the PPG signal as a whole. All three motion classes can have the same impact on the reliability or each motion class can have an individual impact on the reliability of the PPG signal.

Heart rate variation determination unit 60 determines a heart rate based on the optical feature, such as the interbeat intervals of the PPG signal, and evaluates a variation of the heart rate of a subject based on the reliability inputted from signal reliability determination unit 50. In this embodiment, heart rate variation determination unit 60 evaluates the variation of the heart rate to detect AF episodes and to determine AF likelihood based on a time series of IBIs from the PPG signal and the reliability of the respective signal features, i.e. IBIs, depending on the motion of the subject. Since signal reliability can be adjusted for the motion influence period, estimates of AF episodes can be improved and the risk for a false positive classification can be minimized, leading to an improved evaluation of the variation of the heart rate.

FIG. 2 exemplarily shows an example of evaluation system 1 in which casing 6 is attached by means of a wrist belt 7 to a wrist 5 of the subject. Wrist 5 of the subject is a preferred location for positioning evaluation system 1, since subjects are used to carrying wrist worn devices and since it is a very little obtrusive position for such devices. In other words, evaluation system 1 can be worn on wrist 5 all day long, without causing substantial discomfort or the like to the subject. Nevertheless, it is well known to a person skilled in the art that evaluation system 1 or casing 6, respectively, can also be attached to other parts of the subject.

In one example, also two evaluation systems 1 can be attached to both wrists of the subject. By placing two of these systems at two symmetrical human body parts, the monitoring time can be increased by choosing the sensor as signal source, which detects lower motion, i.e. for which the signal reliability determination unit can determine a higher reliability. Disposing in one example two PPG sensors coupled with two motion sensors, preferably accelerometers, at opposite and symmetrical body parts allows increasing the monitoring time by switching between the PPG signals of the two sensors according to the motion level of the respective motion signal. Further, positioning the two PPG sensors at the same but opposite body location of the subject allows developing a single classification system, since the same input type can be employed which alternatively uses data from the two optical sensors. This can further increase the chance of detecting AF events or episodes. Finally, such solution can also allow the detection of AF episodes during intensive physical activities and is thus valuable for determining the most appropriate medical treatment, since the PPG sensor detected by one sensor has good quality for only half the signal period and switching between the two sensors would allow doubling the time with good quality signal features, such as IBIs.

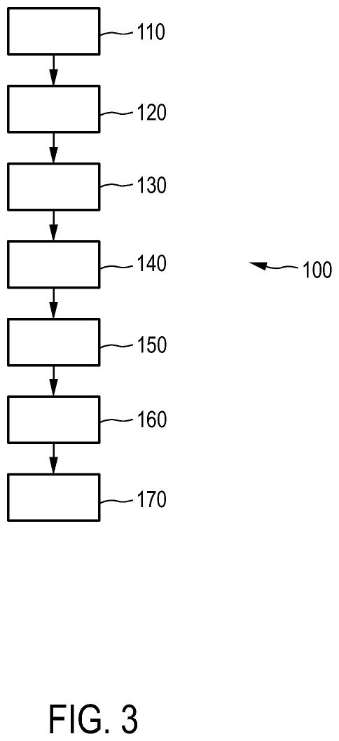

FIG. 3 schematically and exemplarily shows a flow chart of an embodiment of an evaluation method 100 for evaluating a variation of a heart rate of a subject. In step 110, a PPG signal indicative of a heartbeat of the subject is provided. The PPG signal can be obtained in real time or can be stored and provided for later analysis.

In step 120, a motion signal indicative of a motion of the subject is provided. Also the motion signal can be obtained in real time or be a previously stored motion signal. The PPG signal and the motion signal preferably correspond to each other such that they are indicative of the same time period and the same body part of the subject.

In step 130, a signal feature of the PPG signal is determined. The signal feature is for instance an IBI or a morphology of the PPG signal.

In step 140, a motion period during which motion identifiable in the motion signal corresponds to one of a plurality of predefined motion classes is determined. The motion class can be, in general, any class of motion of the subject which can have an influence on reliability of the PPG signal or the signal feature. In this example, the predefined motion classes are i) an activity of the subject, ii) a posture change of the subject or iii) motion of adjacent limbs of the subject, such as the hand or fingers in case the PPG signal and or the motion signal of the subject are related to a wrist of the subject.

In step 150, a reliability of the PPG signal is determined depending on whether a period of the PPG signal is within or outside a motion influence period. The motion influence period consists of the motion period and a transition period which follows the motion period. The transition period accounts for a time period during which the earlier motion still has some influence on the reliability of the PPG signal, but during which, however, no motion in accordance to any of the motion classes is identifiable in the motion signal.

In step 160, a heart rate is determined based on the signal feature. In the example of IBIs, the heart rate is for instance determined by employing statistical algorithms on a time series of the IBIs. Further, the determined reliability can also be taken into account, such that for periods with a low or no reliability, no heart rate can be determined from the PPG signal.

In step 170, a variation of the heart rate of the subject is evaluated based on the determined reliability of the PPG signal. Together, episodes of the PPG signal will less likely be erroneously detected as AF episodes.

Although the above steps are listed in a particular order, these steps can also be carried out in a different order and all the steps can be carried out simultaneously and/or continuously. For example, in case of a wearable monitoring unit which employs evaluation method 100, subjects wearing this monitoring unit can be monitored throughout the day and night for a long period of time and variation of the heart rate can be continuously tracked onset. Further, since a PPG signal instead of, for instance, an electrocardiography (ECG)-based signal, is employed, wearing such monitoring unit is less obtrusive for the subject.

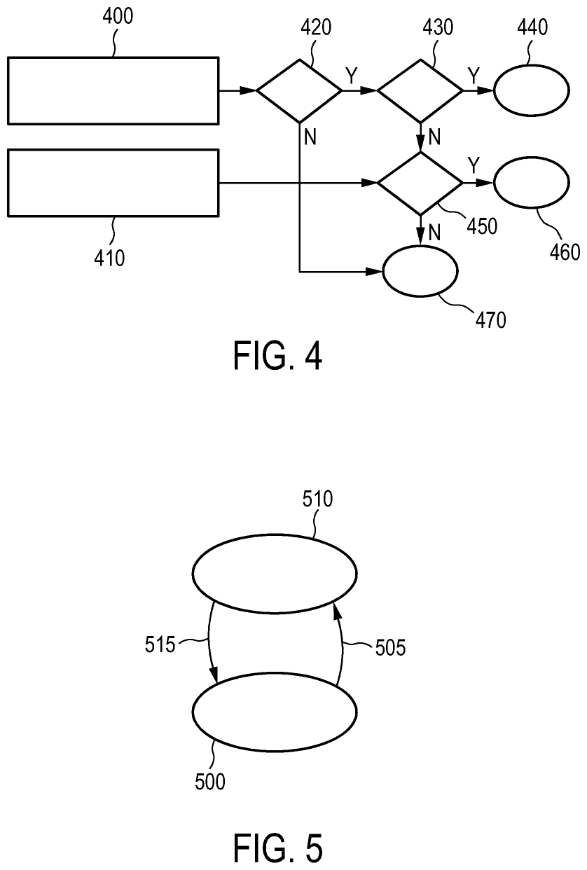

FIG. 4 exemplarily illustrates a first example of a scheme for assessing a reliability of a PPG signal. The system employs motion sensor output 400, which is indicative of the activity type, such as walking, running, cycling, sports, sedentary, etc. In particular, motion output 400 comprises the intensity of motion. Further, the motion signal comprises adjacent parts motion influence 410, which shows motion intensity and motion features indicative of motion of adjacent parts of the subject. Adjacent parts are, for instance, body parts adjacent to the parts a motion sensor is located at or the motion signal originates from, for instance, fingers and the hand in case of a wrist-worn device.

At step 420 it is decided, whether based on motion sensor output 400 the type of activity is changed. In case no change of activity type is detected in step 420, it is continued with step 470 and the previous reliability of the previous signal feature is maintained. In the contrary, in case in step 420 a changed activity is detected, it is further determined in step 430, whether large movement has been detected. In the affirmative, in case large movement has been detected in step 430, the current signal feature, for instance IBI, is discarded in step 440. In the alternative, in case no large movement has been detected in step 430, it is decided in step 450, based on adjacent parts motion in step 410, whether adjacent parts are moving. In case motion of adjacent body are moving. In case motion of adjacent body parts is detected in step 450, reliability is lowered in step 460. In the alternative, in case no motion of adjacent body parts is detected in step 450, the previous reliability of the optical features is maintained in step 470.

A second example of a scheme for assessing a reliability of a PPG signal is illustrated with reference to FIG. 5. FIG. 5 illustrates the exemplary scheme as a state machine with two states, “Use IBI” state 500 and “Discard IBI” state 510. In state 500, the signal feature, such as the IBI, which is currently determined from the PPG signal is used for further evaluation. In the alternative state 510, the current IBI is discarded, i.e. not used for further evaluation. In other words, signal reliability determination unit 50 (cf. FIG. 1) determines whether condition 505 or condition 515 is present and sets the system in accordance therewith to state 500, i.e. to use the IBI, or state 510, i.e. to discard the IBI. Condition 505 corresponds to a motion signal in which an activity type different from sedentary is detected or a posture change is detected or adjacent motion is detected. Condition 515 corresponds to a state in which for the motion influence period comprising the motion period and the transition period, the activity type has been determined to be sedentary, no posture change has been detected and no adjacent motion of adjacent body parts has been detected. In other words, the detection of any of the events “activity type other than sedentary, posture change and motion of adjacent body parts” will cause the state machine to go to “Discard IBI” state 510 and absence of these events for their respective influence periods causes the state machine to be in “Use IBI” state 500.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For example, although a PPG sensing unit in contact with the skin has been mainly disclosed in the description as being the PPG signal providing unit, the same invention holds for other optical signal providing units, like a laser speckle sensing unit, or PPG signal providing units not in contact with the skin, like a vital signs camera. Next to that, more than one PPG signal providing unit may be used.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the determination of a signal feature of the PPG signal, the determination of the motion period of the motion signal, the determination of the reliability of the PPG signal, the determination of heart rate based on the signal feature, the evaluation of the variation of the heart rate of the subject et cetera performed by one or several units or devices can be performed by any other number of units or devices. These procedures and/or the control of the system for evaluating a variation of a heart rate of a subject in accordance with the method for evaluating a variation of a heart rate of a subject can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The present invention relates to a detection of physiological signals of cardiovascular systems. In particular, it relates to a system and method for more reliably evaluating a variation of a heart rate of a subject. The system comprises a PPG signal providing unit (10); a motion signal providing unit (20); a motion determination unit (40) for determining a motion period during which motion identifiable in the motion signal corresponds to one of a plurality of predefined motion classes; a signal reliability determination unit (50) for determining a signal reliability of the PPG signal depending on a motion influence period, wherein the motion influence period comprises the motion period and a transition period following the motion period; and a heart rate variation determination unit (60) for determining the variation of the heart rate of the subject based on the signal reliability and on the PPG signal.

1. A system for evaluating a variation of a heart rate of a subject, comprising:
a photoplethysmography signal providing unit for providing a photoplethysmography signal indicative of a heartbeat of the subject;
a motion signal providing unit for providing a motion signal indicative of a motion of the subject;
a signal feature determination unit for determining a signal feature of the photoplethysmography signal, wherein the signal feature comprises at least one of an interbeat interval and a morphology of the photoplethysmography signal;
a motion determination unit for determining a motion period during which motion identifiable in the motion signal corresponds to one of a plurality of predefined motion classes;
a signal reliability determination unit for determining a signal reliability of the photoplethysmography signal depending on whether a period of the photoplethysmography signal is within or outside a motion influence period, wherein the motion influence period comprises the motion period and a transition period following the motion period; and
a heart rate variation determination unit for determining the variation of the heart rate of the subject based on the signal reliability and on the signal feature of thephotoplethysmography signal.

2. The system according to claim 1, wherein the motion determination unit is adapted to determine a motion class of the plurality of predefined motion classes for the motion period based on the motion signal, wherein the transition period has a length based on the motion class.

3. The system according to claim 1, wherein the motion signal is indicative of a motion of a body part of the subject, and wherein the plurality of predefined motion classes comprises i) an activity of the subject, ii) a posture change of the subject and iii) a movement of an adjacent body part.

4. The system according to claim 1, wherein the motion signal providing unit comprises at least one of a vibration sensor, a rotation sensor and an acceleration sensor for sensing a vibration, a rotation and/or an acceleration of the subject.

5. The system according to claim 1, wherein the motion signal providing unit comprises a network of two or more sensors to be provided at different body parts of the subject.

6. The system according to claim 1, wherein the heart rate variation determination unit is adapted to determine the heart rate of the subject based on the signal reliability and on the signal feature and to determine the variation of the heart rate of the subject based on the heart rate of the subject.

7. The system according to claim 1, wherein the signal reliability determination unit is adapted to determine a binary reliability or a reliability having intermediate values.

8. The system according to claim 1, wherein the signal reliability determination unit is adapted to determine a higher reliability of the photoplethysmography signal outside the motion influence period than within the motion influence period.

9. The system according to claim 1, wherein the system is implemented as a wrist based device.

10. The system according to claim 1, comprising:
a second photoplethysmography signal providing unit for providing a second photoplethysmography signal indicative of the heartbeat of the subject;
a second motion signal providing unit for providing a second motion signal indicative of a motion of the subject;
a second motion class determination unit for determining a second motion period during which motion identifiable in the second motion signal corresponds to one of the plurality of predefined motion classes;
a second signal reliability determination unit for determining a second signal reliability of the second photoplethysmography signal depending on whether a period of the second photoplethysmography signal is within or outside a second motion influence period, wherein the second motion influence period comprises the second motion period and a second transition period following the second motion period;
wherein the heart rate variation determination unit is arranged for determining the variation of the heart rate of the subject based on the signal reliability, the second signal reliability, the photoplethysmography signal and/or the second photoplethysmography signal.

11. The system according to claim 10, wherein the photoplethysmography signal and the motion signal are indicative of a first part of the subject, wherein the second photoplethysmography signal and the second motion signal are indicative of a second part of the subject, wherein the first part and the second part are two symmetrical body parts of the subject.

12. The system according to claim 10, wherein the heart rate variation determination unit is adapted to determine the higher one of the signal reliability and the second signal reliability and to decide on one of the photoplethysmography signal and the second photoplethysmography signal based on the higher one of the signal reliability and the second signal reliability.

13. A method for evaluating a variation of a heart rate of a subject, comprising:
providing a photoplethysmography signal indicative of a heartbeat of the subject;
providing a motion signal indicative of a motion of the subject;
determining a signal feature of the photoplethysmography signal, wherein the signal feature comprises at least one of an interbeat interval and a morphology of the photoplethysmography signal;
determining a motion period during which motion identifiable in the motion signal corresponds to one of a plurality of predefined motion classes;
determining a reliability of the photoplethysmography signal depending on whether a period of the photoplethysmography signal is within or outside a motion influence period, wherein the motion influence period consists of the motion period and a transition period following the motion period;
determining a heart rate based on the signal feature; and
evaluating a variation of the heart rate of the subject based on the determined reliability of the photoplethysmography signal.

14. A computer program for evaluating a variation of a heart rate of a subject, the computer program comprising program code means for causing an evaluation system as defined in claim 1, when the computer program is run on the evaluation system.
